(19) United States
(12) Patent Application Publication
(10) Pub. No.: US 2009/0263494 A1
(43) Pub. Date: Oct. 22, 2009

(54) Manzanita

(76) Inventor: Robert David Emerson, Jupiter, FL (US)

Correspondence Address: Robert Emerson, 405 Philadelphia Drive, Jupiter, FL, 33458, US

(21) Appl. No.: 12/148,498

(22) Filed: Apr. 21, 2008

(51) Int. Cl. A61K 35/54; A61K 36/00; A61P 31/12; A61K 33/32

(52) U.S. Cl. 424/581; 424/775; 424/642

(57) ABSTRACT

Applying the bark of the mariposa Manzanita shrub either mixed into a hot tea or added to mayonnaise and zinc for open sores will cure the herpes virus.

The invention is removing the bark of the manzanita shrub and churning it into a powder for medical applications against the herpes virus, the shingles virus, and herpes cold sores. This can be taken as a tea, pill, or capsule. There are six subspecies of the manzanita shrub:
Manzanita elegans
Manzanita glavcescens
Manzanita manzanita
Manzanita roofii
Manzanita wieslanderi

Also, a salve can be made using the manzanita bark powder, adding an egg white compound and zinc. This salve can be made for treating actual breakouts on the human skin.

1. The bark of the manzanita shrub roughly grated can be made into a pill, capsule, or best made into a hot tea. This will kill the herpes, shingles virus. The pill, capsule, or tea should be taken three times a day. The tea range can be as little as 1% manzanita bark powder and 99% water. It can also be as much as 99% manzanita bark powder and 1% water.

2. A salve can be made for a herpes, shingles or herpes cold sore outbreak. Simply grate the bark of the mariposa manzanita shrub to a fine powder. Mix with any egg white compound such as mayonnaise. Also add zinc. The composition of the salve can range from 1% to 90% mariposa manzanita bark powder. Add to this ½% to 3% zinc. Also add ½% to 30% egg white compound. This will kill the herpes virus breakout on the skin of the person with the affliction. It will kill the shingles breakout on the skin of the person with the affliction. It will also kill the herpes cold sore on the skin of the person with the affliction.
